(19) United States
(12) Patent Application Publication
(10) Pub. No.: US 2003/0163849 A1
(43) Pub. Date: Aug. 28, 2003

(54) Plant gene targeting using oligonucleotides

(76) Inventors: Gregory D. May, Ardmore, OK (US); Eric B. Kmiec, Landenberg, PA (US); Michael C. Rice, Newtown, PA (US)

Correspondence Address: FISH & NEAVE, 1251 AVENUE OF THE AMERICAS, 50TH FLOOR, NEW YORK, NY, 10020-1105, US

(21) Appl. No.: 10/294,172

(22) Filed: Nov. 13, 2002

Related U.S. Application Data
(63) Continuation of application No. PCT/US01/16152, filed on May 17, 2001.
(60) Provisional application No. 60/205,597, filed on May 17, 2000.

(51) Int. Cl. A01H 1/00; C12N 15/82

(52) U.S. Cl. 800/288; 435/468

(57) ABSTRACT

Methods and compositions are presented for the generation of targeted alterations in a plant genome using double-stranded homogeneous oligonucleotides containing a single type of nucleotide. These methods can be used to correct mutations, introduce mutations and/or alter gene activity in a plant cell. A cell-free assay system for monitoring genetic alteration using the oligonucleotides of the invention is also presented.

### TECHNICAL FIELD OF THE INVENTION

The invention relates to gene repair or modification in plants.

### BACKGROUND OF THE INVENTION

Chimeric RNA/DNA oligonucleotides (chimeras) have been used to direct single base changes in episomal and chromosomal targets in mammalian cells (Yoon, et al. 1996. “Targeted gene correction in mammalian cells mediated by a chimeric RNA/DNA oligonucleotide, Proc Natl Acad Sci USA 93: 2071-2076; Cole-Strauss, et al. 1996. “Correction of the mutation responsible for sickle cell anemia directed by a chimeric RNA/DNA oligonucleotide,” Science 273: 1386-1389; Kren, et al. 1998. “In vivo site-directed mutagenesis of the factor IX gene by chimeric RNA/DNA oligonucleotides,” Nature Med 4: 1-6; Alexeev, V. and Yoon, K. 1998. “Stable and inheritable changes in genotype and phenotype of albino melanocytes induced by an RNA-DNA oligonucleotide,” Nature Biotech 16:1343-1346; and Lai, L. -W. and Lien, Y. -HH. 1999. “Homologous recombination-based gene therapy,” Exp Neph 7: 11-14). The process by which these nucleotide conversions are made is still undefined, but recent evidence suggests that mismatch repair plays a critical role in mammalian cells. Using cell-free extracts from HuH7 cells, Cole-Strauss et al. demonstrated that both point and frameshift mutations can be corrected by these chimera and that the reaction is reduced significantly in extracts that lack a functional mismatch repair system (Cole-Strauss, et al. 1999. “A mammalian cell-free extract that directs chimeric RNA/DNA oligonucleotide-mediated gene targeting,” Nucl Acids Res 27: 1323-1330). In addition, antibodies directed against hmsh2, the human homolog of the MutS protein from E. coli, significantly decrease the efficiency of the chimera-based reaction.

While a large body of information exists for bacterial, yeast and mammalian DNA repair systems, there is a paucity of experimental evidence for defining similar reactions in plant cells (Britt, A. B. 1996. “DNA damage and repair in plants,” Ann Rev Plant Physiol Plant Mol Biol 45: 75-100), despite the fact that DNA repair processes impact broad areas of basic and applied plant research, including the control of cell cycle and aspects of recombination. This is due, in part, to plant model systems being less genetically tractable than more thoroughly studied organisms.

As an effective DNA repair system in plants, chimeric RNA/DNA oligonucleotide molecules have been shown to mediate single base changes in plant cells (Zhu, et al. 1999. “Targeted manipulation of maize genes in vivo using chimeric RNA/DNA oligonucleotides,” Proc Natl Acad Sci USA 96: 8768-8773; and Beetham, et al. 1999. “A tool for functional plant genomics: chimeric RNA/DNA oligonucleotides cause in vivo gene-specific mutations,” Proc Natl Acad Sci USA 96: 8774-8778). Zhu et al. reported site-specific heritable GFP mutations in maize genes engineered by introducing chimeric RNA/DNA oligonucleotides into cultured maize cells as well as immature embryos via particle bombardment. While the frequency of site-specific targeting was higher than frequencies of spontaneous mutation and gene targeting by homologous recombination in plants, it was much less than the frequencies found for chimeric RNA/DNA oligonucleotide repairs in mammalian cells. Moreover, while the predicted DNA change was obtained in about 85% of the clones, alternative mutations occurred in adjacent bases. Beetham et al. carried out similar studies using electroporation and particle bombardment to deliver chimeric RNA/DNA oligonucleotides to tobacco Nt-1 cells, thereby conferring herbicide resistance in tobacco cells. In this system, the site of the observed modified base was found to be always in the targeted codon, however, it was shifted one nucleotide 5′ of the target mismatched nucleotides.

In a commentary on gene therapy in plants, Hohn and Puchta (Hohn, B. and Puchta, H. 1999. “Gene therapy in plants,” Proc Natl Acad Sci USA 96: 8321-8323) point out that specific chimeric RNA/DNA oligonucleotides have been used to induce point mutations in several mammalian genes and that chimeric oligonucleotide-dependent mismatch DNA repair has been used in plants (tobacco and maize). A tobacco tissue culture cell line, a cultured maize line, and immature maize embryos have been treated with chimeric oligonucleotides using microparticle bombardment. Delivery of the chimeric oligonucleotide to plants cells was reported to be difficult due to the relatively rigid plant cell wall, resulting in low transformation frequencies. Moreover, inconsistent genetic alteration of the plant cell DNA was noted. With the tobacco cell line, DNA repair was shifted from the expected second position of the target codon to the first position. Likewise, in maize, the target codon as well as the codon 5′ to it was changed.

To date, all gene repair using oligonucleotides has been accomplished with RNA/DNA chimeras. In fact, the concept of chimeric oligonucleotides for gene repair or gene targeting relies on the presence of RNA in the molecule, and recent evidence has confirmed the importance of RNA regions in stabilizing the conjunction of the chimera with the target site (Gamper, H., unpublished). All DNA oligonucleotides, referred to as DNA hairpins, have been tested for gene repair without success. In mammalian cells, various workers have reported that DNA hairpins (i.e., DNA oligonucleotides) could not repair mutations (Yoon, et al. 1996. Proc Natl Acad Sci USA 93: 2071-2076; Cole-Strauss, et al. 1996. Science 273: 1386-1389; and Kren, et al. 1998. Nature Med 4: 1-6).

Methods of targeted gene repair in plants using all-DNA oligonucleotides, all-RNA oligonucleotides, all-PNA oligonucleotides, other oligonucleotides containing all of one type of nucleic acid mimetic, or mixtures thereof have now been found. An assay has also been found in which cell free extracts from monocotyledonous and dicotyledonous plant species as well as embryonic tissue can be used in conjunction with an all-DNA oligonucleotide, all-RNA oligonucleotide, all-PNA oligonucleotide, any other oligonucleotide containing all of one type of nucleic acid mimetic, or a mixture thereof to direct gene conversions.

### BRIEF DESCRIPTION OF THE DRAWINGS

FIG. 1 depicts the targeted plasmid sequence and the all-DNA oligonucleotide, designated Kan 4021-DNA, designed to repair the indicated mutation. The plasmid contains a point mutation at base 4018. This mutation is in the coding region of a gene that confers antibiotic resistance. The sequence of the wild-type, mutant and converted bases are listed below the DNA oligonucleotide designed to correct the mutation. The targeted base is indicated by an arrow.

### DETAILED DESCRIPTION

In the present invention, an all-DNA oligonucleotide, all-RNA oligonucleotide, all-PNA oligonucleotide, any other oligonucleotides containing all of one type of nucleic acid mimetic, or a mixture thereof is useful to effect targeted gene repair in plants.

In the cell free assay of the present invention, gene conversion such as correction of point mutations or frameshift mutations can be conducted in a biochemically controlled environment within a genetically tractable system. The cell-free assay provides a method by which a cell-free extract from a plant of interest is screened for its ability to support point mutation or frameshift mutaion gene conversion. In general, the cell free assay consists of (1) an in vitro reaction involving a plasmid which contains a gene with a point mutation or a frameshift mutation of interest, an oligonucleotide which is believed to contain the genetic code for correcting the gene mutation in the plasmid, and a cell-free extract taken from the plant of interest and (2) a genetic readout system for determining gene conversion. The demonstration that the cell-free extract supports the correction of a point mutation and/or frameshift mutation indicates that the source plant cells possess the machinery to catalyze correction of either one or both types of mutations. The cell-free assay is also useful for elucidating certain DNA repair pathways in plant cells as well as the identification and characterization of proteins involved in the gene repair process.

To detect gene correction, it is believed that any system known in the art which identifies the correction of point or frameshift mutations in a cell-free environment can be used. Preferably, a system using plasmid molecules containing point or frameshift mutations in the coding regions of antibiotic resistance gene is used.

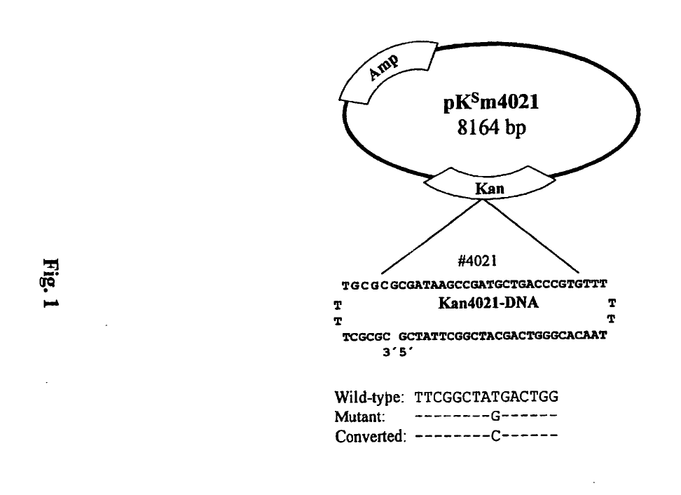

For example, targeted gene repair was accomplished with an all-DNA oligonucleotide, using a cell free extract assay system and a kanamycin-sensitive plasmid to detect site specific repair. The plasmid pK^{S}m4021 contains the mutated kanamycin gene and a wild-type ampicillin resistance gene (FIG. 1). The presence of the ampicillin gene enables control and normalization of the E. coli transformation process. The plasmid and appropriate DNA oligonucleotide are mixed with the extract. After a defined time, the plasmid DNA is extracted and transformed into competent E. coli cells harboring a mutation in the RECA gene. Previous results established the need for functional RecA protein in the bacterial system (Metz, et al. 1998. “Molecular mechanism of chimeric RNA/DNA oligonucleotide directed DNA sequence alteration,” Conference Proceedings: 1^{st} Annual Meeting of the American Society of Gene Therapy, Seattle, Wash., p. 164e). Hence, the use of cells deficient in RecA function ensures that any correction observed after the phenotypic readout had occurred in the cell-free extract. These correction events are scored by selection on agar plates containing kanamycin or tetracycline depending on the plasmid assayed. A dilution from the same transformation was plated in duplicate and selected on plates containing ampicillin to normalize the efficiency of electroporation. Frequencies were calculated as kanamycin/tetracycline revertant colonies relative to ampicillin colonies selected from the same reaction sample.

A final, but important feature of plasmid pK^{S}m4021 is the target sequence itself. Wild-type sequence conferring antibiotic resistance contains a T residue at position 4018. This base was mutated to a G, disabling functional gene activity. To avoid the possibility of positive results emanating from contaminating sources, the DNA oligonucleotide was designed to convert the G residue to a C, instead of a T. This switch still generates a functional protein thereby preserving the phenotypic readout as kanamycin resistance. FIG. 1 illustrates the DNA oligonucleotide used in this study. Kan4021-DNA directs correction, whereas SC1, a non-specific chimera, does not elicit any change.

In this study we used extracts from Musa. Cell-free extracts were prepared using the strategy of Cole-Strauss et al. (Cole-Strauss, et al. 1999. Nucl Acids Res 27: 1323-1330) with slight modifications as outlined in the Methods section. Central among the changes was the use of liquid nitrogen to freeze the samples for grinding with a mortar and pestle. The extract was prepared in 20 mM HEPES (pH 7.5), 5 mM KCl, 1.5 mM MgCl₂, 10 mM DTT, 10% (v/v) glycerol and 1% (w/v) PVP. The extract was mixed with plasmid DNA and the DNA oligonucleotide in a reaction buffer containing NTPs and dNTPs. After incubation, the samples were extracted with phenol/chloroform and precipitated with ethanol. The plasmid DNA was then electroporated into a mutant strain of E. coli, containing a mutation in the RecA gene (DH10B). The bacteria were plated on agar containing the appropriate antibiotic and allowed to grow for 18 hours at 37° C.

Kanamycin resistant colonies are present in samples containing the Musa extracts (data not shown). The conversion required for kan resistance is G→C, and the base pair mismatches created by the DNA oligonucleotide is G/G. This is a purine-purine mismatch and is among the most efficiently repaired, as judged by mammalian cell experiments (Lahue, et al. 1989. “DNA mismatch correction in a defined system,” Science 245: 160-164; Holmes, et al. 1990. “Strand-specific mismatch correction in nuclear extracts of human and Drosophila melanogaster cell lines,” Proc Natl Acad Sci USA 87: 5837-5842). The response was dose-dependent and successful correction relied on the presence of the extract. The maximal frequency of conversion observed in these experiments was approximately 0.08%.

A series of control experiments was performed. Complete reaction mixtures produced colonies, while the absence of plasmid, chimera or extract resulted in no antibiotic resistant colonies (data not shown). Also, the plasmid and the DNA oligonucleotide were incubated separately with the extract, the DNA purified and mixed prior to electroporation. With these reaction parameters, no colonies were observed, reinforcing the fact that the measured correction events occurred in the plant cell extract and not in the bacterial cells.

Conversion at the DNA level was measured by sequencing plasmids isolated from antibiotic resistant bacterial colonies. DNA sequence analysis indicated that the kanamycin sensitive mutant base G had been converted to the base, and sequencing of the non-coding strand confirmed that both strands were repaired (data not shown). Hence, these results suggest that the change from antibiotic sensitivity to antibiotic resistance is the result of a unique nucleotide exchange at position 4021 (kan^{r}).

The concept of gene repair using the chimeric oligonucleotide relies on the presence of RNA in the molecule. Recent evidence has confirmed the importance of this RNA region in stabilizing the conjunction of the chimera with the target site (Gamper et al., submitted). To test the activity of an all-DNA oligonucleotide, we utilized the Musa cell-free extract as it has routinely demonstrated the highest level of repair activity. The oligonucleotide Kan4021-DNA was effective in correcting the mutation in pK^{S}m4021 (see FIG. 1). The action of the DNA oligonucleotide produced antibiotic resistant colonies (Table I). Colonies were selected, the plasmid DNA extracted and the sequence analyzed. Six of 16 colonies from the reaction containing the DNA oligonucleotide harbored plasmid molecules with the targeted sequence alteration. The other 10 colonies contained altered sequence variations (Table I). Hence, 37.5% of the colonies tested contained plasmids with targeted base changes, while 62.5% of the colonies tested contained plasmids with non-targeted base changes.

TABLE I
DNA sequence analyses of pK^{S}m4021 corrections
directed by all-DNA oligonucleotides

| Oligonucleotide | Conversion Type^{a} | Number Observed |
|---|---|---|
| KAN4021-DNA | TAG→TAC | 6 |
| | TAG→CAG | 5 |
| | TAG→TGG | 2 |
| | TAG→TAT | 1 |
| | TAG→TTG | 2 |

^{a}Types (and numbers) of DNA sequence changes directed by an all-DNA oligonucleotide. Underlined nucleotides represent targeted→observed changes, respectively.

This invention describes the use of DNA oligonucleotide “hairpins” for correction of mutations in cell-free extracts from plants. By using mutant strains of E. coli lacking RecA protein activity as a genetic readout system, the results establish sustained inheritance and clonal expansion of corrected DNA templates. Sequence analyses of these clones confirm genetic repair at the DNA level.

Degeneracy in targeted correction was observed when an all-DNA oligonucleotide, designed to adopt the same double hairpin configuration as the chimera, was used to convert the kanamycin mutation in Musa cell free extracts. Over 60% of the isolated plasmid molecules had a variety of altered bases within the specific codon. Based on the design of the genetic readout system, only non-targeted changes that enable antibiotic resistance will be observed. Sequencing 200 bases upstream or downstream from the targeted codon revealed no non-specific, non-targeted mutations. We cannot, however, rule out such mutagenic behavior on plasmids that would not confer kanamycin resistance. This second type of mutagenic activity may be a function of the all-DNA oligonucleotide rather than a property of a particular type of plant extract. Contrary to previous work in the mammalian cells reporting that DNA hairpins could not repair mutations (Yoon, et al. 1996. Proc Natl Acad Sci USA 93: 2071-2076; Cole-Strauss, et al. 1996. Science 273: 1386-1389; and Kren, et al. 1998. Nature Med 4: 1-6)., these results indicate DNA hairpins can be used to repair mutations, evidencing the presence of potentially different repair pathways in plants.

The cell free assay system of the present invention offers several advantages over cell-based methods known in the art. By preparing cell-free extracts from various staged cells, the assay can be used to determine whether the rate of successful targeting is influenced by a particular cell cycle phase. The rate of random mutagenesis to gene conversion can be determined using the assay of the present invention, providing a means to optimize the selection of target plant tissue and the oligonucleotide for gene conversion studies. The assay of the present invention can be used to assess whether a given plant tissue has sufficient enzymatic machinery to catalyze the reactions necessary for gene conversion, thus assisting in the selection of tissue targeted for gene conversion. Using fractionation and biochemical purification methods, the cell free extracts can be analyzed to identify the types of DNA repair proteins present in a given plant cell. Optimum cell culture conditions for gene conversion can be determined by measuring the effect of modification(s) of growth conditions to the rate of gene conversion. The effects of environmental stimuli and the molecular components associated with such a response can be assessed using the assay of the present invention. Characterization of mutant plant lines as well as the molecular basis for certain mutations can also be assessed using the assay of the present invention.

Additional aspects and advantages of the present invention will be described in the following example, which should be regarded as illustrative and not limiting the scope of the present application.

### EXAMPLE 1

### Use of DNA Oligonucleotide to Correct Mutation in Cell Free Extract

Plant Materials

Musa acuminata (banana) cv Rasthali cell suspensions (the kind gift of T. R. Ganapathi) were maintained as shaker cultures (27° C., 80 rpm in a 125 ml flask) and transferred every 10 days to fresh M2 cell suspension medium (Cote, et al. 1996. “Embryogenic cell suspensions from the male flower of Musa AAA cv. Grand Nain,” Physiol Plant 97: 285-290). Dense Musa cell suspensions were centrifuged in 50 mL disposable centrifuge tubes at 700 g for five minutes at room temperature. Following centrifugation, the liquid medium was decanted, and the pelleted cells were frozen in liquid nitrogen and stored at −80° C.

Preparation of Cell-Free Extracts

Cell-free extracts were prepared from Musa cell suspensions by a modification of Cole-Strauss et al. (Cole-Strauss, et al. 1999. Nucl Acids Res 27: 1323-1330). Plant samples were ground under liquid nitrogen with a mortar and pestle. Next 3 mL of the ground plant tissue were extracted in 1.5 mL of extraction buffer (20 mM HEPES, pH 7.5, 5 mM KCl, 1.5 mM MgCl₂, 10 mM DTT, 10% [v/v] glycerol, and 1% [w/v] PVP). Samples were then homogenized with 15 strokes of a Dounce homogenizer. Following homogenization, samples were incubated on ice for 1 hour and centrifuged at 3000 g for 5 min to remove plant cell debris. Protein concentrations of the supernatants were determined by Bradford assay. Extracts were dispensed into 100 μg aliquots, frozen in a dry ice-ethanol bath and stored at −80° C.

Kanamycin selectable marker was used in a substitutory system to determine nucleotide exchange in the cell-free extract. The kanamycin sensitive plasmid pK^{S}m4021 contains a single base transversion (T→G), which creates a TAG stop codon in the kanamycin (kan) gene at codon 22. The plasmid also contains a wild-type ampicillin gene used for propagation and normalization ((Cole-Strauss, et al. 1999. Nucl Acids Res 27: 1323-1330).

Oligonucleotides

Synthetic oligonucleotides were used to direct reversion of a kan^{S} gene to restore resistance to the antibiotic. An all-DNA oligonucleotide, Kan4021-DNA, which can direct conversion of the kan^{S} gene in pK^{S}m4021 at codon 22 from TAG to TAC (stop codon→tyrosine), was synthesized as previously described ((Cole-Strauss, et al. 1999. Nucl Acids Res 27: 1323-1330). The non-specific chimera SC1 (Cole-Strauss, et al. 1996. Science 273: 1386-1389) was used as a control.

In vitro Assays

Reaction Conditions

Reaction mixtures consisted of 1 μg of substrate plasmid pK^{S}m4021 and 1.4 μg of the all-DNA molecule, Kan40211-DNA for kan^{S} system. These components were mixed in a buffer of 20 mM Tris, pH 7.6, 15 mM MgCl₂, 1 mM DTT, 0.2 mM spermidine, 2.5 mM ATP, 0.1 mM each CTP, GTP, UTP, 0.01 mM each dNTPs, 0.1 mM NAD, and 10 μg/mL BSA. The reaction was initialized by adding plant cell-free extracts to 0.1 to 0.8 mg/mL in 100 μL volumes. The reactions were incubated at 30° C. for 1 hour and stopped by placing on ice. The substrate plasmid was then isolated by phase partition with phenol, one chloroform extraction, followed by ethanol precipitation on dry ice for 1 hour and centrifugation at 4° C. for 30 min.

Electroporation, Plating and Selection

Five microliters of resuspended reaction precipitates were used to transform 20 μL aliquots of electrocompetent DH10B bacteria using a Cell-Porator apparatus (Life Technologies) as described by the manufacturer. Each mixture was transferred to a 1 ML SOC culture, incubated at 37° C. for 1 hour, and then converted plasmids were amplified by adding kanamycin to 50 μg/mL and an additional incubation for 3 hours at 37° C. 100 μL aliquots of undiluted cultures were then plated onto LB agar plates containing 50 μg/mL kanamycin. 100 μL aliquots of a 10⁴ dilution of the cultures were also plated onto LB agar plates containing 100 mg/mL ampicillin. Plating was performed in duplicate using sterile Pyrex beads. Both sets of plates were incubated for 16 to 18 hours at 37° C., and colonies were counted using an Accucount 1000 plate reader (Biologics). Targeted conversion of the kan^{S} gene was determined by normalizing the number of kanamycin resistant colonies by dividing by the number of ampicillin resistant colonies, since all plasmids contain a wild type amp gene. Resistant colonies were confirmed by selecting isolated clones for mini preparation of plasmid DNA followed by sequencing using an ABI Prism kit on an automated ABI 310 capillary sequencer.

We claim:

1. A method of altering a plant cell genomic DNA comprising
introducing a DNA oligonucleotide into said plant cell, wherein said DNA oligonucleotide directs at least one single base pair change in a target sequence of said genomic DNA.

2. A method of correcting a mutation in a plant cell genomic DNA comprising
introducing a DNA oligonucleotide into said plant cell, wherein said DNA oligonucleotide directs at least one single base pair change in a target sequence of said genomic DNA, said base pair change restoring said genomic DNA to wild type.

3. A method of inducing a mutation in a plant cell genomic DNA comprising
introducing a DNA oligonucleotide into said plant cell, wherein said DNA oligonucleotide directs at least one single base pair change in a target sequence of said genomic DNA.

4. A method of inactivating an enzyme in a plant cell comprising
introducing a DNA oligonucleotide into said plant cell, wherein said DNA oligonucleotide directs at least one single base pair change in a target sequence of said genomic DNA, said base pair change disrupting the coding region for said enzyme.

5. A method of modifying the bioactivity of an enzyme in a plant cell comprising
introducing a DNA oligonucleotide into said plant cell, wherein said DNA oligonucleotide directs at least one single base pair change in a target sequence of said genomic DNA, said base pair change altering the coding region for said enzyme.

6. A method of modifying a protein in a plant cell comprising
introducing a DNA oligonucleotide into said plant cell, wherein said DNA oligonucleotide directs at least one single base pair change in a target sequence of said genomic DNA, said base pair change disrupting the coding region for said protein.
